(19) United States
(12) Patent Application Publication
(10) Pub. No.: US 2020/0211709 A1
(43) Pub. Date: Jul. 2, 2020

(54) METHOD AND SYSTEM TO PROVIDE MEDICAL ADVICE TO A USER IN REAL TIME BASED ON MEDICAL TRIAGE CONVERSATION

(71) Applicant: MedWhat.com Inc., Spring Hill, FL (US)

(72) Inventor: Arturo DEVESA, Spring Hill, FL (US)

(73) Assignee: MedWhat.com Inc., Spring Hill, FL (US)

(21) Appl. No.: 16/234,304

(22) Filed: Dec. 27, 2018

(51) Int. Cl. G16H 50/30; G16H 10/60; G06F 16/27

(52) U.S. Cl. CPC G16H 50/30; G06F 16/27; G16H 10/60

(57) ABSTRACT

The present disclosure provides a system for providing medical advice to a user in real time based on medical triage conversation. The system creates a health profile of the user based on past records and data entered by the user. The system receives the user enquiry from a communication device. In addition, the system tokenizes the user enquiry into tokens and converts the tokens into word embedding. Further, the system maps the graph of user enquiry created from the word embedding with graph in the corpus of medical triage conversation. Furthermore, the system selects the one or more relevant answers for the user enquiry based on the mapping and the health profile of the user. Moreover, the system display the one or more relevant answers to the user input based on the selection of the one or more relevant answers.

### TECHNICAL FIELD

The present disclosure relates to the field of medical advice, and in particular, relates to a method and system to provide medical advice to a user in real time based on medical triage conversation.

### INTRODUCTION

Over the last few years, there has been increase in the number of diseases to which humans are getting infected. In order to get diagnosed for disease, patient visits a medical practitioner for consultation. The medical practitioners provide consultation to the patient based on symptoms of the patient by providing medicine or diagnosis. The medical practitioners charge fees from the patient for a single consultation or visit. Due to hefty fees of the medical practitioners, the patient looks for medical information which is present on the internet. The information present on the internet is spread over whole digital area and information is extracted based on the grammar used by the patient. The information extracted for the patient is not always the intended information which is relevant for the patient as being extracted based on the grammar and not on the context of the patient.

### SUMMARY

In a first example, a computer-implemented method is provided. The computer-implemented method to provide a user with one or more relevant answers based on user enquiry. The computer-implemented method may include a first step to create health profile of the user based on past records and data entered by the user. The computer-implemented method may include a second step to receive a user enquiry from a communication device. In addition, the computer-implemented method may include a third step to tokenize the user enquiry into tokens. Further, the computer-implemented method may include a fourth step to convert the tokens into word embedding. Furthermore, the computer-implemented method may include a fifth step to map the graph of user enquiry created from the word embedding with graph in the corpus of medical triage conversation. Moreover, the computer-implemented method may include a sixth step to select one or more relevant answers for the user enquiry. The one or more relevant answers are selected from the corpus of the medical training dataset based on the mapping and the health profile of the user. The computer-implemented method may include a seventh step to display the one or more relevant answers to the user enquiry based on the selection of the one or more relevant answers. The tokenization is done to convert text string of the user enquiry into the tokens. The tokenization is done in real time. The conversion is done by converting the tokens into one-hot vector representation which is than fed to recurrent neural network and tanh is applied in real time. The conversion is done to achieve user embedding of the user enquiry. The mapping is done to identify similar word embedding of the user enquiry in the corpus of medical training dataset related to the user embedding. The one or more relevant answers are correct answer for the user enquiry being selected from the corpus of the medical triage conversation. The one or more relevant answers selected comply with one or more medical protocols for the user enquiry. The one or more relevant answers are displayed in real time on the communication device.

In an embodiment of the present disclosure, the corpus of the medical triage conversation may include a plurality of question-answer pairs, a plurality of medical questions, a plurality of medical articles and a plurality of medical conversations. The corpus of the medical triage conversation is created from one or more sources. The one or more sources may include medical literature, textbooks, online databases, journal articles, graphics, podcasts, videos, animations and medical data warehouses.

In an embodiment of the present disclosure, the past record may include medical record, prescription, medical history, medical policy detail, hereditary disease, user allergies and user infections, wherein the past record is from one or more third party databases.

In an embodiment of the present disclosure, the one or more medical protocols may include rules, regulations and guidelines to provide the medical guidance to the user.

In an embodiment of the present disclosure, the selection of the one or more relevant answers for the user enquiry is done based on confidence level of each of the one or more relevant answers.

In an embodiment of the present disclosure, the health profile of the user may include name, age, demographic information, medical record, prescription, hereditary disease and the like. In an embodiment of the present disclosure, the health profile of the user may include allergies, infections, blood group, hemoglobin level, number of platelets and common symptoms.

In an embodiment of the present disclosure, tokenization may insert delineation tokens into the context and utterances present in the user enquiry. The insertion of the delineation token is done to distinguish the medical assistant system and the user enquiry.

In an embodiment of the present disclosure, the computer implemented method may create the graph of the user enquiry based on the conversion of the user enquiry into word embedding. The graph is created in real time using artificial intelligence algorithm.

In an embodiment of the present disclosure, the computer implemented method may updates the user enquiry after receiving the updated user enquiry from the user. The updating is done based on selection from the one or more relevant answers displayed to the user. The updating is done in real time.

In a second example, a computer system is provided. The computer system may include one or more processors and a memory coupled to the one or more processors. The memory may store instructions which, when executed by the one or more processors, may cause the one or more processors to perform a method. The method to provide a user with one or more relevant answers based on user enquiry. The method may include a first step to receive a user enquiry from a communication device. The method may include a second step to create a health profile of the user based on past record and data entered by the user. In addition, the method may include a third step to tokenize the user enquiry into tokens. Further, the method may include a fourth step to convert the tokens into word embedding. Furthermore, the method may include a fifth step to map the graph of user enquiry created from the word embedding with graph in the corpus of medical triage conversation. Moreover, the method may include a sixth step to select one or more relevant answers for the user enquiry based on mapping and the health profile of the user. The method may include a seventh step to display the one or more relevant answers for the user enquiry based on the selection of the one or more relevant answers. The tokenization is done to convert text string of the user enquiry into the tokens. The tokenization is done in real time. The conversion is done by converting the tokens into one-hot vector representation which is than fed to recurrent neural network and tanh is applied in real time. The conversion is done to achieve user embedding of the user enquiry. The mapping is done to identify similar word embedding of the user enquiry in the corpus of medical training dataset related to the user embedding. The one or more relevant answers are correct answer for the user enquiry being selected from the corpus of the medical triage conversation. The one or more relevant answers selected comply with the one or more medical protocols for the user enquiry. The one or more relevant answers are displayed in real time on the communication device.

In a third example, a non-transitory computer-readable storage medium is provided. The non-transitory computer-readable storage medium encodes computer executable instruction which when executed by at least one processor may perform a method to provide a user with one or more relevant answers based on user enquiry. The method may include a first step to receive a user enquiry from a communication device. The method may include a second step to create a health profile of the user based on past record and data entered by the user. In addition, the method may include a third step to tokenize the user enquiry into tokens. Further, the method may include a fourth step to convert the tokens into word embedding. Furthermore, the method may include a fifth step to map the graph of user enquiry created from the word embedding with graph in the corpus of medical triage conversation. Moreover, the method may include a sixth step to select one or more relevant answers for the user enquiry based on mapping and the health profile of the user. The method may include a seventh step to display the one or more relevant answers for the user enquiry based on the selection of the one or more relevant answers. The tokenization is done to convert text string of the user enquiry into the tokens. The tokenization is done in real time. The conversion is done by converting the tokens into one-hot vector representation which is than fed to recurrent neural network and tanh is applied in real time. The conversion is done to achieve user embedding of the user enquiry. The mapping is done to identify similar word embedding of the user enquiry in the corpus of medical training dataset related to the user embedding. The one or more relevant answers are correct answer for the user enquiry being selected from the corpus of the medical triage conversation. The one or more relevant answers selected comply with the one or more medical protocols for the user enquiry. The one or more relevant answers are displayed in real time on the communication device.

### BRIEF DESCRIPTION OF DRAWINGS

Having thus described the invention in general terms, references will now be made to the accompanying figures, wherein:

FIG. 1 illustrates an example of interaction of the user with the communication device for medical advice, in accordance with various embodiments of the present disclosure;

FIG. 2 illustrates an interactive computing environment to provide medical advice to a user in real time based on medical triage conversation, in accordance with various embodiments of the present disclosure;

FIG. 3 illustrates a block diagram of an example for processing of the user enquiry, in accordance with various embodiments of the present disclosure;

FIG. 4 illustrates a block diagram of an example for implementation of word embedding by medical assistant system, in accordance with various embodiments of the present disclosure;

FIG. 5 illustrates a block diagram for execution of learning of the medical assistant system, in accordance with various embodiments of the present disclosure;

FIG. 6 illustrates a flow chart for depicting internal representation of medical knowledge based dialogue conducted between the medical assistant system and the user, in accordance with various embodiments of the present disclosure;

FIG. 7 illustrates a block diagram of separate RNN's used for computation of margin loss to be minimized during processing of the medical assistant system, in accordance with various embodiments of the present disclosure;

FIG. 8 illustrates an internal representation of tokens and utterances during the medical knowledge based dialogue, in accordance with various embodiments of the present disclosure;

FIG. 9 illustrates a flow chart for depicting a method to provide medical advice to the user in real time based on the medical triage conversation, in accordance with various embodiments of the present disclosure;

FIGS. 10A and 10B illustrate a flow chart to provide medical advice to the user in real time based on the medical triage conversation, in accordance with various embodiments of the present disclosure; and

FIG. 11 illustrates a block diagram of a computing device, in accordance with various embodiments of the present disclosure.

It should be noted that the accompanying figures are intended to present illustrations of exemplary embodiments of the present disclosure. These figures are not intended to limit the scope of the present disclosure. It should also be noted that accompanying figures are not necessarily drawn to scale.

### DETAILED DESCRIPTION

In the following description, for purposes of explanation, numerous specific details are set forth in order to provide a thorough understanding of the present technology. It will be apparent, however, to one skilled in the art that the present technology can be practiced without these specific details. In other instances, structures and devices are shown in block diagram form only in order to avoid obscuring the present technology.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Moreover, although the following description contains many specifics for the purposes of illustration, anyone skilled in the art will appreciate that many variations and/or alterations to said details are within the scope of the present technology.

Similarly, although many of the features of the present technology are described in terms of each other, or in conjunction with each other, one skilled in the art will appreciate that many of these features can be provided independently of other features. Accordingly, this description of the present technology is set forth without any loss of generality to, and without imposing limitations upon, the present technology.

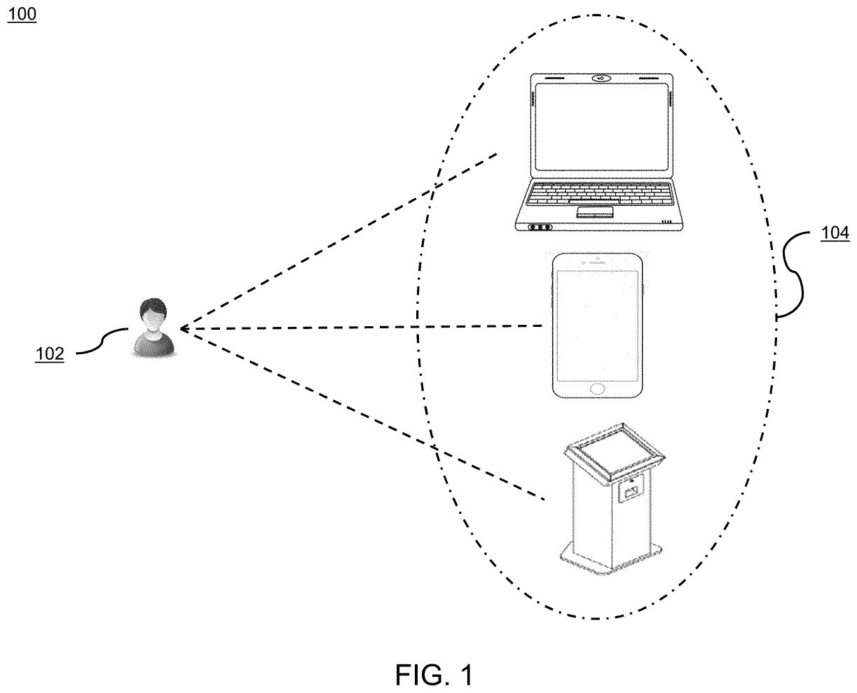

FIG. 1 illustrates a general overview 100 of interaction of a user 102 with a communication device 104 for medical guidance or medical advice, in accordance with various embodiments of the present disclosure. The general overview 100 includes the user 102 and the communication device 104. In an embodiment of the present disclosure, the user 102 is any person who wants medical guidance from a person having medical knowledge. In another embodiment of the present disclosure, the user 102 is any person who wants medical guidance from a medical practitioner. In another embodiment of the present disclosure, the user 102 is any person suffering from some disease. In yet another embodiment of the present disclosure, the user 102 is any person who wants medical guidance or medical advice to cure sickness or to know his disease based on the symptoms. In yet another embodiment of the present disclosure, the user 102 is any person who wants to know severity of the disease or sickness faced by the user 102. In yet another embodiment of the present disclosure, the user 102 requires general medical information to improve his/her knowledge regarding medical subject.

Further, the general overview 100 includes the communication device 104. The communication device 104 is associated with the user 102. In an embodiment of the present disclosure, the communication device 104 is a portable communication device 104. In an example, the portable communication device 104 includes laptop, smartphone, tablet, PDA and the like. In another embodiment of the present disclosure, the communication device 104 is a fixed communication device 104. In an example, the fixed communication device 104 includes a desktop, a workstation PC, a mainframe computer and the like. In yet another embodiment of the present disclosure, the communication device 104 is a kiosk installed at a medical facility. In general, the kiosk is a small, free-standing physical structure that displays information or provides a service to the user 102.

In an embodiment of the present disclosure, the communication device 104 includes an advanced vision display panel. The advanced vision display panel includes

OLED, AMOLED, Super AMOLED, Retina display, Haptic touchscreen display and the like. In another embodiment of the present disclosure, the communication device 104 includes a basic display panel. The basic display panel includes but may not be limited to LCD, IPS-LCD, capacitive touchscreen LCD, resistive touchscreen LCD, TFT-LCD and the like.

In addition, the communication device 104 performs computing operations based on operating system installed inside the communication device 104. In general, the operating system is system software that manages computer hardware and software resources and provides common services for computer programs. In addition, the operating system acts as an interface for software installed inside the communication device 104 to interact with hardware components of the communication device 104. In an embodiment of the present disclosure, the operating system installed inside the communication device 104 is a mobile operating system. In an embodiment of the present disclosure, the communication device 104 performs computing operations based on any suitable operating system designed for the communication device 104. In an example, the mobile operating system includes but may not be limited to Windows operating system from Microsoft, Android operating system from Google, iOS operating system from Apple, Symbian operating system from Nokia, Bada operating system from Samsung Electronics and BlackBerry operating system from BlackBerry. However, the operating system is not limited to above mentioned operating systems. In an embodiment of the present disclosure, the communication device 104 operates on any version of particular operating system of above mentioned operating systems.

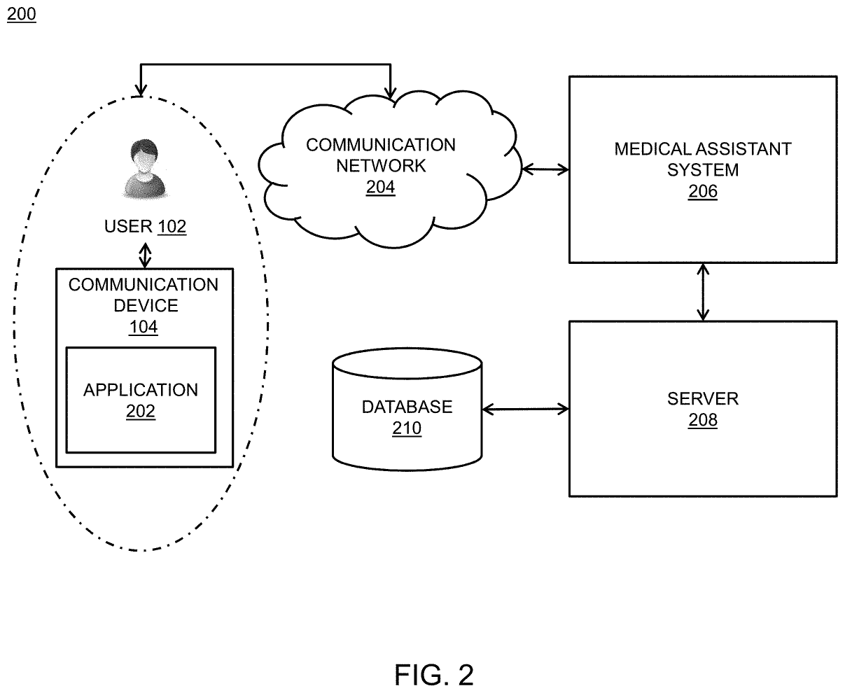

FIG. 2 illustrates an interactive computing environment 200 to provide medical advice to the user 102 in real time based on medical triage conversation. The computing environment 200 includes the user 102, the communication device 104, an application 202, a communication network 204, and a medical assistant system 206. Further, the interactive computing environment 200 includes a server 208 and a database 210.

The interactive computing environment 200 includes the application 202 used for viewing content on the communication device 104. In an example, the application 202 may be one of mobile application, web application or website. The application 202 is the mobile application which displays content to the users 102 on the communication device 104. In general, the application 202 is any software code that is programmed to interact with hardware elements of the computing system to perform one or more step of steps. The term hardware elements here refer to hardware installed inside the communication device 104. Moreover, the application 202 is used to access, read, update and modify data stored in hardware elements of the communication device 104. Also, the application 202 provides a user interface to the user 102 to interact with hardware elements of the communication device 104. The user interface may include Graphical User Interface (GUI), Application Programming Interface (API), and the like. The user interface helps to send and receive user 102 commands and data. In addition, the user interface serves to display or return results of operation from the application 202. In addition, the application 202 installed inside the communication device 104 is based on any platform. In an example, the mobile platform includes but may not be limited to Android, iOS, BlackBerry, Symbian, Windows and Bada. In an embodiment of the present disclosure, the application 202 is used by the user 102 for getting medical guidance. The information is passed from the communication device 104 through the communication network 204.

The interactive computing environment 202 includes the communication network 204. The communication network 204 enables the medical assistant system 206 to transfer information to and from the communication device 104. The communication network 204 is used to connect to the medical assistant system 206. Also, the communication network 204 provides network connectivity to the communication device 104. In an example, the communication network 204 uses protocol for connecting the communication device 104 to the medical assistant system 206. The communication network 204 connects the communication device 104 to the medical assistant system 206 using 2G, 3G, 4G, Wifi and the like.

In an embodiment of the present disclosure, the communication network 204 may be any type of network that provides internet connectivity to the communication device 104. In an embodiment of the present disclosure, the communication network 204 is a wireless mobile network. In another embodiment of the present disclosure, the communication network 204 is a wired network connection. In yet another embodiment of the present disclosure, the communication network 204 is combination of the wireless and the wired network for optimum throughput of data transmission.

The interactive computing environment 200 includes the medical assistant system 206. The medical assistant system 206 is used to provide medical advice to the user in real time based on medical triage conversation. The medical assistant system 206 processes the data received from the user 102 in order to provide the one or more relevant answers to the user 102 based on user context. In an embodiment of the present disclosure, the medical assistant system 206 is installed inside the communication device 104.

The medical assistant system 206 provides an medical knowledge based dialogue for the user 102 on the communication device 104. The user utilizes the communication device 104 to interact with the medical assistant system 206 for medical guidance or medical advice. In an example, the user 102 is suffering from running nose and headache, the user 102 search the website and interacts with the medical assistant system 206. The medical knowledge based dialogue is a bi-directional conversation between the medical assistant system 206 and the user 102 through the communication device 104 in real-time. In general, the medical knowledge based dialogue is a session in which communication takes place efficiently between the user 102 and the system. In an embodiment of the present disclosure, the medical assistant system 206 enquires the first question from the user 102. In an example, the medical assistant system 206 enquires the user 102 with question such as “How are you feeling today?” In another embodiment of the present disclosure, the user 102 initially enquires a question from the medical assistant system 206. The medical knowledge based dialogue is initialized to facilitate the medical assistant system 206 to provide the medical guidance or the medical advice to the user 102.

The medical assistant system 206 creates a health profile of the user 102. The health profile of the user 102 is created based on past records and data entered by the user 102 on the communication device 104. The past records includes but may not be limited to medical record, prescription, medical history, medical policy detail, hereditary disease, user allergies and user infections. The past records are collected from one or more third party databases. The one or more third party databases include medical institution databases, hospital databases, insurance databases, doctor databases and the like. In an embodiment of the present disclosure, the medical assistant system 206 connects with the one or more third party databases in order to collect health related data of the user 102. In an embodiment, the health profile of the user 102 is created based on a plurality of factors. The plurality of factors include health related information associated with the user 102, disease prevalence of the user 102, lifestyle of the user 102, environmental conditions of the user 102, socioeconomic conditions affecting the user 102, medical reports associated with the user 102, previous interaction of the user 102 with the medical assistant system 206 and the like. In an embodiment of the present disclosure, the health profile of the user 102 is created based on a set of data collected from one or more health related devices associated with the user 102. In an embodiment, the health profile of the user 102 includes health related information associated with the user 102 such as name, age, sex, demographic information, blood group, hemoglobin level, number of platelets and the like. In another embodiment, the health profile of the user 102 is created based on the symptoms of the user 102. In yet another embodiment, the health profile of the user 102 includes re-occurrence of disease to the user 102 after an interval of time.

In an embodiment of the present disclosure, the medical assistant system 206 collects data entered by the user 102 for creating the health profile of the user 102. The medical assistant system 206 collects the data such as medical reports associated with the user 102, socioeconomic conditions affecting the user 102 and the like to create the health profile of the user 102. In addition, the medical assistant system 206 is connected with the one or more health related devices associated with the user 102. In an example, the one or more health related devices include activity trackers, smart watches, smartphones, wearable and the like. The medical assistant system 206 fetches the set of data associated with health status of the user 102 from the one or more connected health related devices. The medical assistant system 206 fetches the set of data from the one or more connected health related devices to create the health profile of the user 102. In an embodiment of the present disclosure, the medical assistant system 206 determines health status of the user 102 based on the set of data fetched from the one or more connected health related devices. The set of data fetched is stored in the health profile associated with the user 102. The medical assistant system 206 initializes a concept identification module to parse text present in the medical knowledge based dialogue. Further, the concept identification module retrieves specific concept from the medical knowledge based dialogue. In an embodiment of the present disclosure, the concept identification module parses text to create the word embedding. In another embodiment of the present disclosure, the concept identification module parses the text to create the word embedding. The concept identification module is initialized to identify context of the medical knowledge based dialogue. The concept identification module is initialized to determine if the specific concept match with one or more medical protocols. Further, the medical assistant system 206 represents the retrieved specific concept as word embedding in the low dimensional vector space. In addition, the medical assistant system 206 represents the retrieved specific concept as word embedding in the low dimensional vector space.

The one or more medical protocols include but may not be limited to rules, regulations and guidelines to provide medical guidance to the user 102. The one or more medical protocols are guidelines from one or more medical institutions to provide medical guidance to the user 102. In general, the one or more medical protocols are guidelines or rules to be followed for proper treatment of a patient.

The medical assistant system 206 receives a user enquiry from a communication device 104. The user enquiry is received when the user interact with the medical assistant system 206 using the application 202 of the communication device 104. The user enquiry is received when the user 102 enters or input on the communication device 104 through the graphical user Interface (GUI) of the communication device 104.

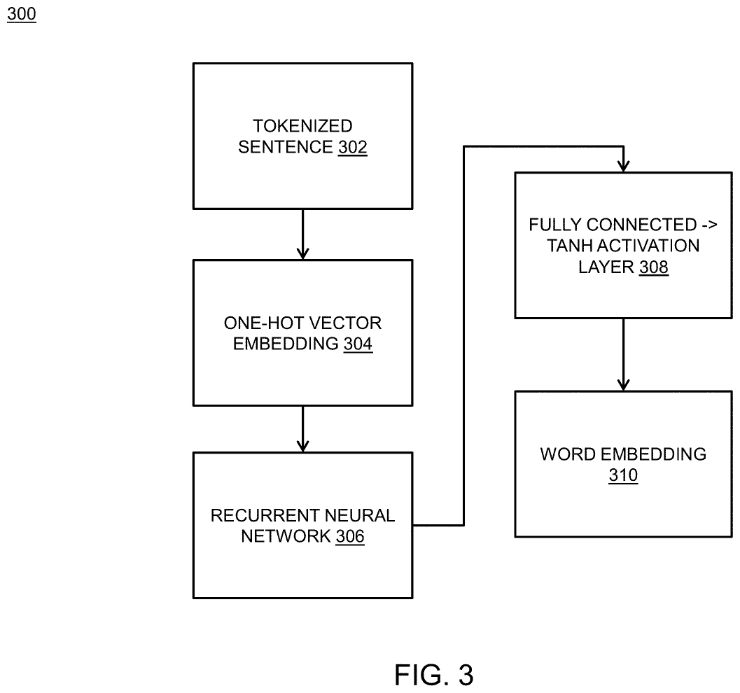

Reference in the embodiment will now be made to the components mentioned in FIG. 3 in order to explain the processing of the medical assistant system 206. FIG. 3 is a block diagram of an example for processing of the user enquiry in accordance with various embodiment of the present disclosure.

In addition, the medical assistant system 206 tokenizes the user enquiry into tokens. The tokenization is done to convert text string of the user enquiry into the tokens. In general, the tokenization is the process of converting the text string of the user enquiry into integers. In an embodiment of the present disclosure, the medical assistant system 206 tokenizes the context and utterances to insert special delineation tokens between the context and utterances. The medical assistant system 206 inserts the special delineation tokens to distinguish between responses of the medical assistant system 206 and the user 102. The tokenization is done in real time. The medical assistant system 206 tokenizes the contexts and utterances based on length of the contexts and utterances. In an embodiment of the present disclosure, the length of token for question or context is specified to be of 30 tokens. However, the length of token for contexts may be changed accordingly. In an embodiment of the present disclosure, the length of token for utterances is specified to be of 70 tokens. However the length of token for utterances may be changed accordingly. In an embodiment of the present disclosure, the loss percentage is kept at less than 2%. However, the loss percentage is not fixed at 2%. In an embodiment of the present disclosure, the medical assistant system 206 discards the tokenized contexts having length greater than 30 tokens. In an embodiment of the present disclosure, the medical assistant system 206 discards the tokenized utterances having length greater than 70 tokens. In addition, the medical assistant system 206 adds zero-padding to the pairs of contexts and utterances that are less than 30 tokens and 70 tokens respectively. The tokenization is done and tokenized sentence 302 is received after the tokenization of the user enquiry by the medical assistant system 206.

In addition, the medical assistant system 206 performs conversion of the tokenized sentence 302 of the user enquiry into word embedding. The tokenized sentence 302 is converted into word embedding by converting the tokens into one-hot vector representation. The conversion of the tokens into one-hot vector embeddings 304 is done by the medical assistant system 206. In general, the one-hot vector representation is a way of vector representation in which all elements of the vector are zero except one, which has one as its value. Further, the list of one-hot vectors representation is fed to recurrent neural network (RNN) 306. In an embodiment of the present disclosure, the medical assistant system 206 uses long short term memory (hereinafter, LSTM) as the recurrent neural network 306. In general, the LSTM units are units of the recurrent neural network. In general, the LSTM unit is composed of a cell, an input gate, an output gate and a forget gate. Further, the cell remembers values over arbitrary time intervals and the three gates regulate the flow of information into and out of the cell. However, the medical assistant system 206 is not limited to using LSTM as the recurrent neural network.

In addition, the recurrent layer of the recurrent neural network 306 is applied with dropout to prevent over fitting. In general, the dropout is a regularization technique for neural networks where randomly selected neurons are ignored during training. In general, the over fitting is a modeling error which occurs when a function is too closely fit to a limited set of data points. Furthermore, the output of the recurrent neural network 306 at last timestamp is fed to a fully connected layer with tanh nonlinearity as shown in FIG. 3 as fully connected tanh activation layer 308. In general, the tanh is applied interpreted as a probabilistic function as the output of the tanh function ranges from 0 to 1. In general, the fully connected layers connect every neuron in one layer to every neuron in another layer. The output represents word embedding of the user enquiry as shown in FIG. 3 as word embedding 310.

Further, the medical assistant system 206 creates graph of the user enquiry based on the conversion of the user enquiry into the word embedding. The graph is created in real time using artificial intelligence algorithm. The graph of the user enquiry is created based on the context of the user 102. The graph of the user enquiry shows the relation between the words used by the user 102 in the user enquiry present in the word embedding. The graph of the user enquiry is created for every word embedding in real time.

Furthermore, the medical assistant system 206 maps the graph of the user enquiry with the graph present in a corpus of medical triage conversation. The comparison is done to identify similar word embedding of the user enquiry in the corpus of medical training dataset related to the user enquiry. The mapping of the graph of the user enquiry is done to identify the graph with same structure of the user enquiry in the corpus of medical training dataset. The mapping of the word embedding of the user enquiry with the word embedding of words present in the corpus of medical triage conversation include mapping the user enquiry with the questions present in a plurality of dialogue conversations. The plurality of dialogue conversations are conversations between a plurality of users and a plurality of professional medical practitioners. The plurality of dialogue conversations are extracted from the corpus of medical triage conversation using natural language processing algorithms and speech recognition algorithms. In an embodiment of the present disclosure, the plurality of dialogue conversations is converted into machine-usable form using decision trees. In general, the decision tree is a decision support tool that uses a tree-like graph or model of decisions and their possible consequences, including chance event outcomes, resource costs, and utility. In an embodiment of the present disclosure, the plurality of dialogue conversations is converted into machine-usable form using hardware-run deep learning algorithms. In another embodiment of the present disclosure, the plurality of dialogue conversations is used to assess level of severity of condition of the user 102. The level of severity of condition of the user 102 facilitate whether the user 102 requires emergency medical attention, primary care, home care and the like.

In an embodiment, the medical assistant system 206 prioritizes the plurality of dialogue conversations extracted from the corpus of medical triage conversation based on the one or more medical protocols. In an embodiment of the present disclosure, the medical assistant system 206 automatically prioritizes the plurality of extracted dialogue conversations using the hardware-run deep learning algorithms. In another embodiment of the present disclosure, the plurality of extracted dialogue conversations is prioritized manually in the medical assistant system 206. The prioritization is done to train the medical assistant system 206 to learn an appropriate utterance to respond to the user 102 from the corpus of medical triage conversation. The plurality of dialogue conversations extracted is prioritized based on a confidence score. The confidence score determines amount of confidence in the plurality of answers to reply as the one or more relevant output to the user 102.

In an embodiment of the present disclosure, the mapping is done by extracting the graph present in the corpus of medical triage conversation based on the word embedding of the user enquiry. The graphs extracted from the corpus of medical training dataset are one which are prioritized from the plurality of dialogue conversations. The mapping is performed based on the context of the medical knowledge based dialogue using unsupervised deep learning algorithms. In an embodiment of the present disclosure, the mapping is performed based on the context of the medical knowledge based dialogue using word vector embedding. In an example, the medical assistant system 206 scans through large streams of data present in the corpus of medical triage conversation. Furthermore, the mapping of the word embedding of the retrieved specific concept from the user enquiry with the word embedding of words present in the corpus of medical triage conversation is done. The mapping includes mapping the user enquiry with the one or more relevant answers present in the plurality of dialogue conversations. The mapping is performed based on the context of the user enquiry using supervised deep learning algorithms. In an example, the RNN (conversational network) is trained to predict the appropriate utterance as response to the user enquiry by the user 102. The medical assistant system 206 is trained to learn to interpret semantic information present in the user enquiry to map with the appropriate utterance or relevant answer to the user enquiry. The medical assistant system 206 interprets semantic information present in the user enquiry and maps it with the data present in the corpus of medical training dataset.

In an embodiment, the mapping is done to determine the appropriate utterance as response to the user enquiry in context of the medical knowledge based dialogue. The context is concatenation of the user enquiry as well as past utterances by the user 102 and the medical assistant system 206. The mapping is done to determine the appropriate utterance as response even when the word embedding of the retrieved specific concept from the user enquiry is not present in the word embedding of words created from the corpus of medical triage conversation using deep learning algorithms and neural network models.

The corpus of medical triage conversation includes but may not be limited to a plurality of question-answer pairs, a plurality of medical questions and a plurality of medical articles. In an embodiment of the present disclosure, the corpus of medical triage conversation includes a plurality of medical conversations, a plurality of medical facts and the like. The corpus of medical triage conversation is created from one or more sources. Further, the one or more sources include medical literature, textbooks, online databases, journal articles, graphics, podcasts, videos, animations, medical devices and medical protocols. The corpus of medical triage conversation is present in a plurality of input forms. The plurality of input forms include but may not be limited to at least one of text, image, audio, video, gif and animation.

In an embodiment of the present disclosure, the corpus of medical triage conversation includes the plurality of question-answer pairs. The plurality of question-answer pairs are extracted from the one or more sources. In an embodiment of the present disclosure, a plurality of answers of the plurality of question-answer pairs is generated by medical practitioners. In an embodiment of the present disclosure, the plurality of answers of the plurality of question-answer pairs is generated by layperson. In an embodiment of the present disclosure, the plurality of question-answer pairs is utilized as reserve resource in the corpus of medical triage conversation.

In an embodiment of the present disclosure, the corpus of medical triage conversation includes the plurality of medical questions. In an embodiment of the present disclosure, the plurality of medical questions does not have answers for the plurality of medical questions. In another embodiment of the present disclosure, the plurality of medical questions does have answers associated with the plurality of medical questions. The plurality of medical questions is extracted from the one or more sources. In an example, the plurality of medical questions does not provide relevant answers for the plurality of medical questions.

In an embodiment of the present disclosure, the corpus of medical triage conversation includes the plurality of medical articles. The plurality of medical articles is extracted from the one or more sources. The plurality of medical facts is used to train the medical assistant system 206 for continuation of bi-directional conversation between the user 102 and the medical assistant system 206. In another embodiment of the present disclosure, the corpus of medical triage conversation includes the plurality of medical conversations extracted from the one or more sources.

Moreover, the medical assistant system 206 selects the one or more relevant answers for the user enquiry based on mapping and the health profile of the user 102. The one or more relevant answers include the severity of the user to see the medical practitioner based on the analysis of the user 102 symptoms. In an embodiment of the present disclosure, the one or more relevant answers are correct answer for the user enquiry being selected from the corpus of medical triage conversation. In an embodiment of the present disclosure, the one or more relevant answers are the set of questions to be displayed to the user for providing the correct answer for the user enquiry. The selection of the one or more relevant answers for the user enquiry is done based on confidence level of each of the one or more relevant answers. The one or more relevant answers selected comply with the one or more medical protocols for the user enquiry. The confidence level is the probability of the one or more relevant answers being the correct answer or reply to the user enquiry. If the confidence level for one of the one or more relevant answers is high based on the user enquiry than the medical assistant system 206 consider it as the correct answer to the user enquiry. In an example, the plurality of questions from the plurality of dialogue conversations is mapped to determine the most relevant utterance as the one or more relevant answers to the user 102 for the user enquiry.

The selection of the one or more relevant answers from the corpus of medical triage conversation is done by creating word embedding of words present in the health profile of the user 102. In another embodiment of the present disclosure, the medical assistant system 206 creates word embedding of sentences present in the health profile of the user 102. In an embodiment of the present disclosure, the word embedding of words present in the health profile of the user 102 is represented in the low dimensional vector space. In an embodiment of the present disclosure, the concept identification module retrieves specific concept from the health profile of the user 102. In an embodiment of the present disclosure, the concept identification module parses text present in the health profile of the user 102 to create the word embedding. In another embodiment of the present disclosure, the concept identification module parses text present in the health profile of the user 102 to create the word embedding.

In an embodiment of the present disclosure, the medical assistant system 206 interactively replies the user 102 with the one or more relevant output based on the health profile of the user 102. In an example, the medical assistant system 206 analyzes various medical records, data from the one or more medical devices, and the like to create the health profile of the user 102. Further, the medical assistant system 206 utilizes the training data as well as the analyzed data from the health profile of the user 102 to respond to the user enquiry by the user 102. In an embodiment of the present disclosure, the medical assistant system 206 analyzes the health profile of the user 102 to continue the medical knowledge based dialogue between the medical assistant system 206 and the user 102. The medical assistant system 206 continues the medical knowledge based dialogue with the user 102 based on the determined health status of the user 102. In addition, the medical assistant system 206 responds to the user enquiry of the user 102 based on the data associated with the health profile of the user 102. In an example, the health profile of user X shows that the user X is suffering from diabetes. Further, the user X enquires the user enquiry related to diabetes from the medical assistant system 206.

In an embodiment of the present disclosure, the medical assistant system 206 creates sentence embedding of entire sentences in the plurality of dialogue conversations. In an embodiment of the present disclosure, the plurality of dialogue conversations are represented as decision trees based on the one or more medical protocols. The sentence embedding of entire sentences is created by embedding entire sentences in the low-dimensional vector space. Further, the sentence embedding is used in sequence to sequence network (also termed as encoder decoder architecture) to produce end-to-end trainable neural translation models. In an embodiment of the present disclosure, the decoder uses global embedding to generate natural language from different language that was embedded by the decoder. In an embodiment of the present disclosure, the sentence embedding is generated using recurrent neural networks, convolution neural networks, and combinations thereof.

In an embodiment of the present disclosure, the recurrent neural network is trained in such a manner that similarity measure of two matching embedding is maximal. In addition, the recurrent neural network is trained in such a manner that similarity measure of two mismatched embedding is minimal. The recurrent neural network is trained using a ranking loss function to achieve above stated objectives during training of the recurrent neural network. In an embodiment of the present disclosure, the word embedding for the user enquiry is considered to be as x_{Q}. In an embodiment of the present disclosure, the word embedding for true answer of the user enquiry is considered to be as x_{QA}. In an embodiment of the present disclosure, the word embedding for false or incorrect answer of the user enquiry is considered to be as x_{FA}. Thus, the ranking loss function is defined as:

L(x_{Q}; x_{TA}; x_{FA})=max (0, (M−(s(x_{Q}; x_{TA})−s(x_{Q}, x_{FA}))))

In addition, the cosine-similarity is used as measure s(·, ·) and defined as s(x,y)=(x·y)/(∥x∥·∥y∥). In addition, M is margin parameter. The aim is to minimize the above stated loss to encourage margin between correct and incorrect pairings of the embedding. Further, the similarity for correct pairings is at least M larger than similarity of incorrect pairing. In addition, a single training input includes a question, its correct answer, and a randomly chosen incorrect answer. In an embodiment of the present disclosure, the medical assistant system 206 utilizes embedding functions for embedding context pairs using the ranking loss function. Furthermore, the medical assistant system 206 utilizes embedding functions for embedding context and utterance pairs using the ranking loss function for preparing the one or more relevant answers.

Also, the medical assistant system 206 display the one or more relevant answers for the user enquiry based on the selection of the one or more relevant answers. The one or more relevant answers are displayed on the application 202 of the communication device 104. In an embodiment of the present disclosure, the one or more relevant answers displayed on the application 202 are question for the user 102. In another embodiment of the present disclosure, the one or more relevant answers include report, suggestion, guidance to the user 102 based on the symptoms of the user 102. In an embodiment, the medical assistant system 206 interactively replies to the user enquiry in a plurality of output forms. The plurality of output forms include at least one of text, image, audio, video, gif and animation.

Also, the medical assistant system 206 updates the user enquiry after receiving the updated user enquiry from the user 102 based on selection from the one or more relevant answers displayed to the user 102. The updating of the user enquiry is done when the medical processing system 206 reply to the user enquiry with the one or more relevant answers in form of question. The user 102 replies to the one or more relevant answers with a new set of the user enquiry which is received at the medical assistant system 206. The updating is done in real time.

In an embodiment, the medical assistant system 206 learns from the past decision of the medical assistant system 206. The learning of the medical assistant system 206 initially includes crawling the corpus of medical triage conversation to obtain the plurality of the dialogue conversations from the corpus of medical triage conversation. Further, the learning of the medical assistant system 206 includes manual annotation of questions based on a plurality of criteria. In an embodiment of the present disclosure, the plurality of criteria includes user feedback, new content sources, popular questions and the like. Further, the manual annotation of questions is followed by binary annotation of the dialogue conversations. Further, the learning of the medical assistant system 206 is continued by building automatic classifiers for prioritizing the plurality of dialogue conversations. The automatic classifiers are built using a plurality of hardware-run machine learning algorithms. The plurality of hardware-run machine learning algorithms includes Random Forest, Adaboost, Naive Bayes, Support Vector Machine algorithm and the like.

The medical assistant system 206 updates the health profile of the user 102 and the corpus of medical triage conversation in real-time using recurrent neural networks. The medical assistant system 206 does updating based on the user enquiry from the user 102 that are not present in the word embedding of words created from the corpus of the medical triage conversation.

In an embodiment of the present disclosure, the medical assistant system 206 is machine learning based medical assistant that enables the medical knowledge based dialogue between the user 102 and the medical assistant system 206. The medical assistant system 206 interactively replies to the user enquiry or the symptoms of the user 102 automatically. In addition, the medical assistant system 206 initializes the medical knowledge based dialogue between the user 102 and the medical assistant system 206. Also, the medical assistant system 206 probe the user enquiry from the user 102 to get better idea about the health status of the user 102. In an embodiment of the present disclosure, the medical assistant system 206 is based on natural language processing algorithms, information retrieval algorithms, knowledge representation algorithms, semantic web algorithms, medical informatics algorithm and the like. In general, the natural language processing is defined as automatic manipulation of natural language such as speech, text, and the like. In general, the information retrieval is the activity of obtaining information system resources relevant to an information need from a collection of information resources. In general, the knowledge representation is the field of artificial intelligence dedicated towards representing information about the world in a form that computer system can utilize to solve complex tasks. In an example, the complex tasks include tasks such as diagnosing a medical condition, having a dialog in a natural language and the like. In general, the semantic web is a proposed development of World Wide Web in which data in web pages is structured and tagged in such a way that it can be read directly by computers. In general, the medical informatics is the intersection of information science, computer science, and health care.

In an embodiment of the present disclosure, the medical assistant system 206 enables the medical knowledge based dialogue between the user 102 and the medical assistant system 206 in one or more languages. The medical assistant system 206 may be trained in any one of the one or more languages. Further, the medical assistant system 206 may respond to the user 102 in specified language of the one or more languages. In an embodiment of the present disclosure, the medical knowledge based dialogue between the user 102 and the medical assistant system 206 is enabled in English language. In another embodiment of the present disclosure, the medical knowledge based dialogue between the user 102 and the medical assistant system 206 is enabled in Hindi language. In yet another embodiment of the present disclosure, the medical knowledge based dialogue between the user 102 and the medical assistant system 206 is enabled in any language of the one or more languages such as Spanish, Hindi, Chinese, Japanese and the like.

In an example, the medical assistant system 206 learns and updates itself dynamically in real-time. The medical assistant system 206 automatically learns to respond to the user 102 based on the user enquiry in real-time. The medical assistant system 206 uses past interaction history of the user 102 as well as the corpus of medical triage conversation to interactively reply the user 102 with the one or more relevant answers. The medical assistant system 206 responds to the user enquiry by the user 102 based on hardware-run machine learning algorithms.

In an example, the user 102 initializes the medical knowledge based dialogue by enquiring to the medical assistant system 206 “I drink alcohol with my malaria pills?” The medical assistant system 206 may enquire the user 102 back such as “What is the name of your malaria pills?” Further, the user 102 responds to the medical assistant system 206 with name of the malaria pills. Furthermore, the medical assistant system 206 may interactively reply back to the user 102 with appropriate one or more relevant answers such as “I would not recommend drinking alcohol with Malarone pills. The pills may cause dizziness which can worsen with alcohol”.

In an embodiment of the present disclosure, the medical assistant system 206 is designed to provide various advantages. The advantage of the medical assistant system 206 is to converse fluently in natural language. In an example, suppose the user 102 enquires the medical assistant system 206 “Should I go see the doctor?” The medical assistant system 206 should interactively reply the user 102 with the appropriate utterance such as “Yes, you should go see the doctor”. The medical assistant system 206 should not reply with something inappropriate such as “My name is Sam”. Another advantage of the medical assistant system 206 is to produce accurate, reliable and appropriate utterances to the user enquiry or the user symptoms from medical point of view. The advantage is achieved by associating each utterance as response to the user 102 with the confidence level. In addition, the second objective of the plurality of objectives is achieved by converting the utterances into fact-based utterances. In an example, the medical assistant system 206 may respond something as “If you have taken Malarone and are experiencing severe dizziness, you should go see your doctor”.

In an embodiment of the present disclosure, the medical assistant system 206 is based on RNN architecture. In general, the recurrent neural networks are neural network models that operate over sequential data and show strong ability for modeling natural language. The RNN-based sequence to sequence models obtain state-of-the-art performance in context of conversations and question-answering. The RNN is used to read an input sequence (the user enquiry or the user symptoms, for example) one word at a time. Further, the RNN encodes the input sequence into a state vector that compresses semantic information. Further, the semantic information is used to initialize the second RNN that decodes the semantic information to produce the one or more relevant answers, output one word at a time. The medical assistant system 206 is trained through reading a plurality of examples of question-answer or statement-response pairs. Further, the medical assistant system 206 determines most similar medical question whose answer has already been trained to the medical assistant system 206. Furthermore, the medical assistant system 206 returns the one or more relevant answers as response corresponding to context of the user enquiry or the user symptoms by the user 102.

In an embodiment of the present disclosure, the medical assistant system 206 achieves performance in Top-1 accuracy and mean reciprocal rank metrics. In general, the Top-1 accuracy is how many times correct label has highest probability predicted by network. In general, the mean reciprocal rank is a statistic measure for evaluating any process that produces a list of possible responses to a sample of queries, ordered by probability of correctness.

In an embodiment of the present disclosure, the GPU-compatible model of the medical assistant system 206 is implemented using Keras library. In an embodiment of the present disclosure, the single-GPU compatible model takes a time period of about 40 minutes to iterate one epoch of about millions of question-answer pairs. In an embodiment of the present disclosure, the medical assistant system 206 computes the word embedding of single question and the plurality of answers present in the plurality of dialogue conversations. The medical assistant system 206 ranks answers by associating the confidence level with the answers. Further, the medical assistant system 206 performs with the top-1 accuracy and mean reciprocal rank metrics. The top-1 accuracy measures fraction of instances where algorithm returns the true answer as its top choice in the ranking. In an example, the medical assistant system 206 performs ranking of 2000 user enquiry. The medical assistant system 206 is asked to rank 200 answers to the user enquiry during each instance. Further, the questions and answers used for this evaluation are held out from training set as validation set. Further, the medical assistant system 206 achieves performance of ˜0.41 mean reciprocal rank and ˜0.25 top-1 accuracy on this validation set.

In an example, the user 102 enquires the medical assistant system 206 with “Do you think I am fat?” The medical assistant system 206 replies interactively to the user 102 with the one or more relevant answers or appropriate utterances with their associated confidence level. The utterance having the most confidence level is denoted as rank 1 and utterance having the least confidence level is denoted as rank 10. In an example, the virtual medical assistant interactively replies as:

“Rank 1.0: “the body uses fat stores when it needs more calories than are taken in this can occur at any age”

Rank 2.0: “and what are you going to do about it I am sure that it can be calculated but the info is completely useless and devoid of any clinical implication you want to burn calories do it the only available way exercise.”

Rank 3.0: “by simply losing 5 to 10 pounds you will reduce the fat.”

Rank 4.0: “watch your calories and avoid hi cholesterol items.”

Rank 5.0: “always non-surgical such as a strict regimen of diet and exercise with a nutritionist on board that way you can keep the weight you lose with the lifestyle modification save abdominoplasty as a last resort best of luck.”

Rank 6.0: “nope you've got it 100 of calories come from either protein fat or carbohydrates the only caloric foods on planet earth.”

Rank 7.0: “you may participate in any con contact sports though low impact exercise like walking swimming will be better have a diet rich in fresh vegetables fruits whole grains nut slow fat milk products beans legumes lentils limit intake of saturated fats no tobacco nor alcohol.”

Rank 8.0: “if u maintain control over your caloric intake.”

Rank 9.0: “you do not qualify for such surgery and risks are real and significant a bariatric program can counsel you regarding diet exercise and eating behaviors.”

Rank 10.0: “a person need not take more than 30 of the calories in the form of fat.”

The answers provided by the medical assistant system 206 become more accurate and precise as the learning of the medical assistant system 206 is continued.

In another example, the user 102 enquires the medical assistant system 206 with “What are the symptoms of the flu?” The medical assistant system 206 replies interactively to the user 102 with the one or more relevant answers or the appropriate utterances with their associated confidence level. The utterance having the most confidence level is denoted as rank 1 and utterance having the least confidence level is denoted as rank 10. In an example, the virtual medical assistant interactively replies as:

Rank 1.0: “the symptoms you describe could be side effects of the amoxicillin she should contact the doctor who prescribed the amoxicillin to determine if a medication change is needed”.

Rank 2.0: “you don't have high fever typical in influenza.”

Rank 3.0: “sometimes amoxicillin can upset your stomach try another antibiotic discuss with your doctor.”

Rank 4.0: “stay as active as you can but at the end of the day get plenty of rest make sure to eat well and stay hydrated take meds to relieve symptoms and wash your hands frequently to prevent spreading this to others report to your doc if you take a sudden turn for the worse or symptoms become severe remember to get flu vaccine every year.”

Rank 5.0: “ask your pcp what is he testing you for the eosinophils are increased in allergic conditions mostly seeing an allergist might be useful.”

Rank 6.0: “there can be more than one flu virus that you want to fend off.”

Rank 7.0: “hopefully you got a flu shot to prevent this kind of infection if not then go get tested for flu and start the appropriate medication.”

Rank 8.0: “no fever would not necessarily be present with the onset of the flu.”

Rank 9.0: “a flu shot should not cause arash.”

Rank 10.0: “some patients with flu have mild illness without fever.”

The answers provided by the medical assistant system 206 become more accurate and precise as the training of the medical assistant system 206 is continued.

In an embodiment of the present disclosure, the medical assistant system 206 is improved in performance and quality based on tuning of one or more hyperparameters. The one or more hyperparameters include but may not be limited to learning rate and associated learning schedule, size and number of recurrent layers, dimensionality of the word embedding, and unsupervised pre-trained weights for the word embedding layer. Further, the one or more hyperparmeters include dimensionality of the sentence embedding layer, dropout and other regularization parameters, type of loss function or margin parameter, the similarity measure and the like. In addition, the medical assistant system 206 is improved in quality by enabling weight-sharing between recurrent networks for questions as well as answers. Also, the medical assistant system 206 is improved in quality by enabling weight-sharing in the word embedding layer.

In an embodiment of the present disclosure, the medical assistant system 206 determines medical issue faced by the user 102. The medical assistant system 206 determines the medical issue by checking for symptoms of diseases entered by the user 102 on the application 202. In an embodiment of the present disclosure, the medical assistant system 206 checks for symptoms of the user 102 based on the health profile of the user 102. In an embodiment of the present disclosure, the medical assistant system 206 checks for symptoms of the user 102 based on one or more hardware-run algorithms.

In addition, the interactive computing environment 200 includes the server 208. In general, the server 208 is a computer program that provides service to another computer programs. In general, the server 208 may provide various functionalities or services, such as sharing data or resources among multiple clients, performing computation for a client and the like. In an example, the server 208 may be at least one of dedicated server, cloud server, virtual private server and the like. However, the server 208 is not limited to above mentioned server 208. In another embodiment of the present disclosure, the medical assistant system 206 is installed at one or more servers. In an example, the plurality of servers may include database server, file server, application server and the like. The server 208 is associated with the database 210.

Further, the interactive computing environment 200 includes the database 210. In general, the database 210 is a collection of information that is organized so that it can be easily accessed, managed and updated. In an example, the database 210 may be one of at least hierarchical database 210, network database 210, relational database 210, object-oriented database 210 and the like. The database 210 provides storage location to the set of data, the health profile of data, the corpus of medical triage conversation, and the like. In an embodiment of the present disclosure, the database 210 provides storage location to all the data and information required by the medical assistant system 206. In an example, the database 210 is connected to the server 208. The server 208 stores data in the database 210. The server 208 interacts with the database 210 to retrieve the stored data.

FIG. 3 illustrates a block diagram 300 of an example for processing of the user enquiry, in accordance with various embodiments of the present disclosure. It may be noted that to explain the process steps of block diagram 300, references will be made to the system elements of FIG. 1 and FIG. 2.

It is shown in FIG. 1 that the user 102 utilizes the communication device 104 to enable medical knowledge based dialogue between the medical assistant system 206 and the user 102; however, those skilled in the art would appreciate that there may be more number of users 102 connecting to the more number of communication device 104.

The medical assistant system 206 may be implemented using the single communication device 104, or a network of communication device, including cloud-based computer implementations. The communication device 104 is preferably server class computers including one or more high-performance computer processors and random access memory, and running an operating system such as LINUX or variants thereof. The operations of the medical assistant system 206 as described herein can be controlled through either hardware or through computer programs installed in non-transitory computer readable storage devices such as solid state drives or magnetic storage devices and executed by the processors to perform the functions described herein. The database 210 is implemented using non-transitory computer readable storage devices, and suitable database management systems for data access and retrieval. The medical assistant system 206 includes other hardware elements necessary for the operations described herein, including network interfaces and protocols, input devices for data entry, and output devices for display, printing, or other presentations of data. Additionally, the operations listed here are necessarily performed at such a frequency and over such a large set of data that they must be performed by a computer in order to be performed in a commercially useful amount of time, and thus cannot be performed in any useful embodiment by mental steps in the human mind.

The block diagram 300 includes the tokenized sentence 302, one-hot vector embedding 304, the recurrent neural network 306, the fully connected->tan h activation layer 308, and the sentence embedding 310. The medical assistant system 206 strips and tokenizes the user enquiry. The tokenized sentence 302 is represented in the form of one-hot vector embedding 304. The one-hot vector embedding 304 representation of each word is fed through an embedding layer. Further, the embedded word representations are fed to the recurrent neural network 306. The output of the recurrent neural network 306 goes through the fully connected->tan h activation layer 308. Furthermore, the output of the fully connected->tan h activation layer 308 is fed to the sentence embedding 310(as described above).

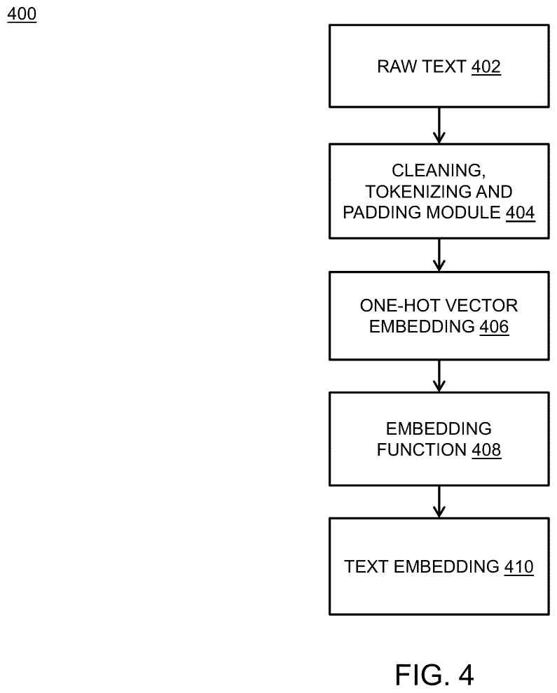

FIG. 4 illustrates a block diagram 400 of an example for implementation of word embedding by the medical assistant system, in accordance with various embodiments of the present disclosure. It may be noted that to explain the process steps of block diagram 400, references will be made to the system components mentioned of FIG. 1 and FIG. 2.

The block diagram 400 includes a raw text 402, a cleaning, tokenizing and padding module 404, one-hot vector embedding 406, an embedding function 408, and a text embedding 410. The raw text 402 is provided to the cleaning, tokenizing and padding module 404. The cleaning, tokenizing and padding module 404 strips and tokenizes the raw text 402. In addition, the cleaning, tokenizing and padding module 404 performs zero-padding to shorter text pieces from the raw text 402. Further, the cleaned, tokenized and padded text is represented in the form of the one-hot vector embedding 406. The one-hot vector embedding 406 representation of each word is passed through the embedding function 408. Further, the embedded word representations are fed to a recurrent neural network. The output of the recurrent neural network goes through a fully connected->tan h activation layer. Furthermore, the output of the fully connected->tan h activation layer represents the text embedding 410.

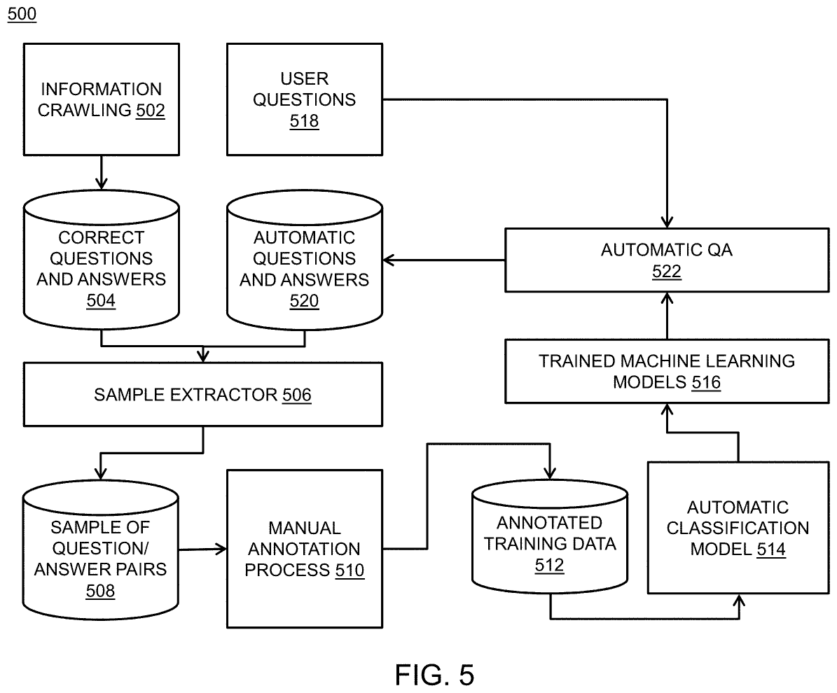

FIG. 5 illustrates a block diagram 500 for execution of learning of the medical assistant system 206 during training, in accordance with various embodiments of the present disclosure. It may be noted that to explain the process steps of block diagram 500, references will be made to the system elements of FIG. 1 and FIG. 2.

The block diagram 500 includes information crawling 502, correct questions and answers 504, a sample extractor 506, a sample of question answer pairs 508, a manual annotation process 510, an annotated training data 512, an automatic classification model 514, a trained machine learning model 516, user questions 518, automatic questions and answers 520, and automatic QA 522. The information crawling 502 refers to crawling of information from the corpus of medical triage conversation. The information crawling 502 is performed to find the correct questions and answers 504 from the corpus of medical triage conversation. Further, the correct questions and answers 504 are indexed by conceptual representation of their titles. In an embodiment of the present disclosure, the concepts are extracted by a hardware-run concept identification algorithm. The correct questions and answers 504 are fed to the sample extractor 506. Further, the sample extractor 506 extracts the sample of question answer pairs 508. The sample of question answer pairs 508 are fed to the manual annotation process 510. The manual annotation process 510 outputs the annotated training data 512. The annotated training data 512 is fed to the automatic classification model 514. In an embodiment of the present disclosure, the automatic classification model 514 trains one or more classifiers that assign the confidence level to the sample of question answer pairs 508. Further, the automatic classification model 514 is connected to the trained machine learning model 516. The trained machine learning model 516 is connected to the automatic QA 522. In addition, the user questions 518 represent the user enquiry by the user 102. The user questions 518 are converted to the automatic questions and answers 520. Further, the automatic questions and answers 520 are fed to the automatic QA 522.

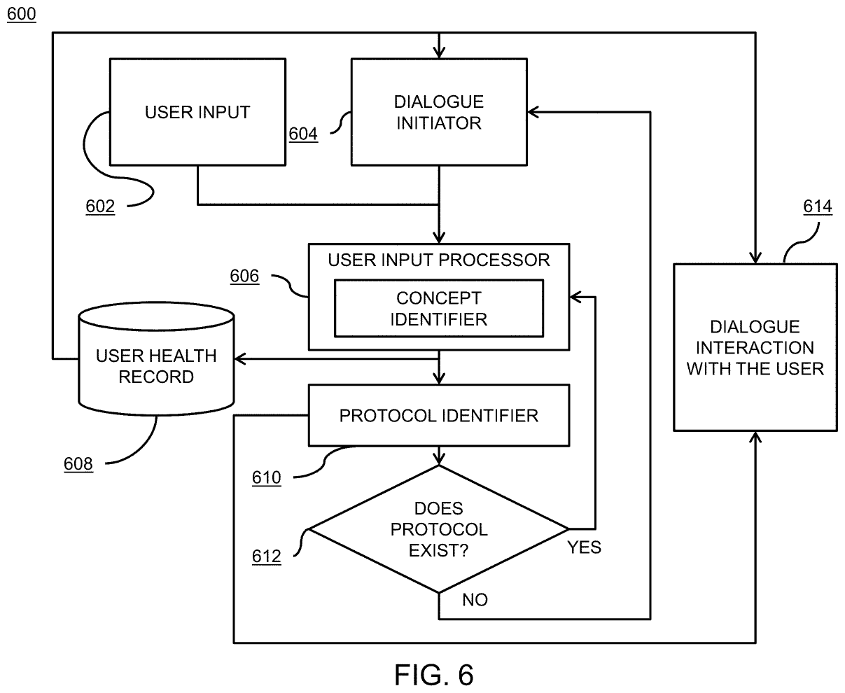

FIG. 6 illustrates a flow chart 600 for depicting internal representation of the medical knowledge based dialogue conducted between the medical assistant system 206 and the user 102, in accordance with various embodiments of the present disclosure. It may be noted that to explain the process steps of flowchart 600, references will be made to the components mentioned in FIG. 1 and FIG. 2.

The flowchart 600 includes a user input 602. The user input 602 is used to take input from the user. In an embodiment of the present disclosure, the user input 602 is used to take the user input from the user 102. The user input 602 is fed to a user input processor 606. In addition, the user input processor 606 includes a concept identifier. In addition, the flow chart 600 includes a dialogue initiator 604. The dialogue initiator 604 initiates the bi-directional conversation with the user 102. The dialogue initiator is converted to a user health record 608. The user health record 608 includes the health profile of the user 102. Further, the user input processor 606 processes input from the user 102. The input from the user 102 may include asking questions from the medical assistant system 206, answering question asked by the medical assistant system 206 and the like. In an embodiment of the present disclosure, the user health record 608 of the user 102 is updated when the user 102 answers the question asked by the medical assistant system 206. In another embodiment of the present disclosure, the concept identification module is applied when the user 102 enquires with new user input. Further, the protocol identifier 610 search for the one or more medical protocols to determine one or more concepts in the user input 604 which matched with the one or more protocols. Further, the medical assistant system 206 checks whether protocol exists or not at a step 612. If the protocol does not exist, the command goes back to the dialogue initiator 604. If the protocol exists, the dialogue interaction with the user 102 is continued until the medical assistant system 206 provides medical guidance to the user 102 at step 614 (as mentioned above).

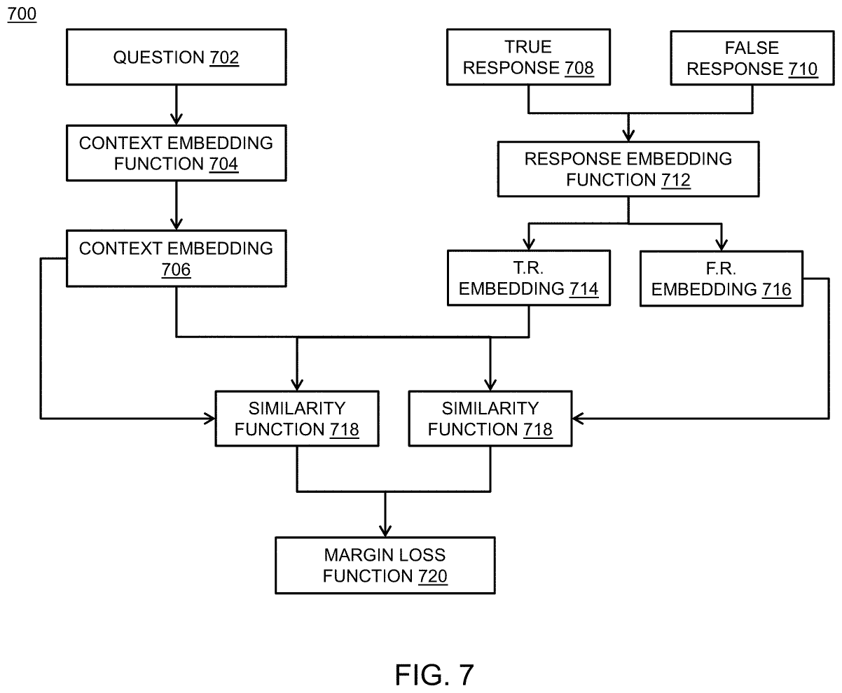

FIG. 7 illustrates a block diagram 700 of separate RNN's used for computation of the margin loss to be minimized during training of the medical assistant system 206, in accordance with various embodiments of the present disclosure. It may be noted that to explain the process steps of block diagram 700, references will be made to the system elements of FIG. 1, and FIG. 2.

The block diagram 700 includes a question 702 that is the user enquiry by the user 102 from the medical assistant system 206. The question passes through a separate RNN for questions. The separate RNN for question is a context embedding function 704. In addition, a response embedding function 712 is a separate RNN for answers. Further, a true response 708 denotes correct answer for the question 702. Furthermore, a false response 710 denotes incorrect answer for the question 702. The word embedding of question is denoted as a context embedding 706. The word embedding for correct answer is denoted as a true response embedding 714. The word embedding for incorrect answer is denoted as a false response embedding 716. Further, the block diagram 700 includes a similarity function 718. In an embodiment of the present disclosure, the similarity function 718 is the ranking loss function. The similarity function 718 maps the embedding of the question 702 with embedding of correct answer. Further, the similarity function 718 gives the confidence score for correct question-answer pair. In addition, the similarity function 718 gives the confidence score for incorrect question-answer pair in a similar manner. (as mentioned above) Furthermore, a margin loss 720 is applied to compute the margin loss. The margin loss 720 appears to be severe when the confidence score of incorrect pairing is not sufficiently much lower than the confidence score of correct pairing. The margin loss function 720 separates the incorrect pairing from the correct pairing. (as mentioned above)

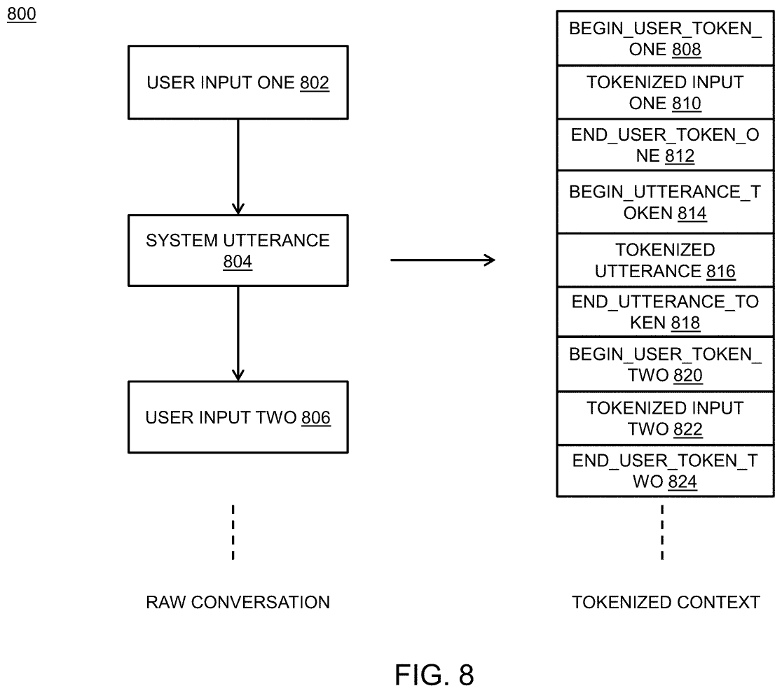

FIG. 8 illustrates an internal representation 800 of tokens and utterances during training of the medical assistant system 206, in accordance with various embodiments of the present disclosure. It may be noted that to explain the internal representation 800, references will be made to the system elements of FIG. 1, and FIG. 2.

The internal representation 800 includes a user input one 802 of the user enquiry. Further, the internal representation 800 includes a system utterance 804. Furthermore, the internal representation 800 includes a user input two 806 of the user enquiry. In addition, the internal representation 800 includes a begin_user_token_one 808. Moreover, the internal representation 800 includes a tokenized input 810. Further, the internal representation 800 includes an end_user_token_one 812. Furthermore, the internal representation 800 includes a begin_utterance_token 814. Also, the internal representation 800 includes a tokenized utterance 816. Moreover, the internal representation 800 includes an end_utterance_token 818. Further, the internal representation 800 includes a begin_user_token_two 820. Furthermore, the internal representation 800 includes a tokenized input two 822. In addition, the internal representation 800 includes an end_user_token_two 824. In an embodiment of the present disclosure, the user input one 802, the system utterance 804, the user input two 806, and the like represents raw conversation. In an embodiment of the present disclosure, the begin_user_token_one 808, the tokenized input 810, the end_user_token_one 812, the begin_utterance_token 814, the tokenized utterance 816, the end_utterance_token 818, the begin_user_token_two 820, the tokenized input two 822, the end_user_token_two 824, and the like represents tokenized context. The medical assistant system 206 inserts the tokens between utterances to differentiate between different utterances between the user and the medical assistant system 206. (as mentioned above)

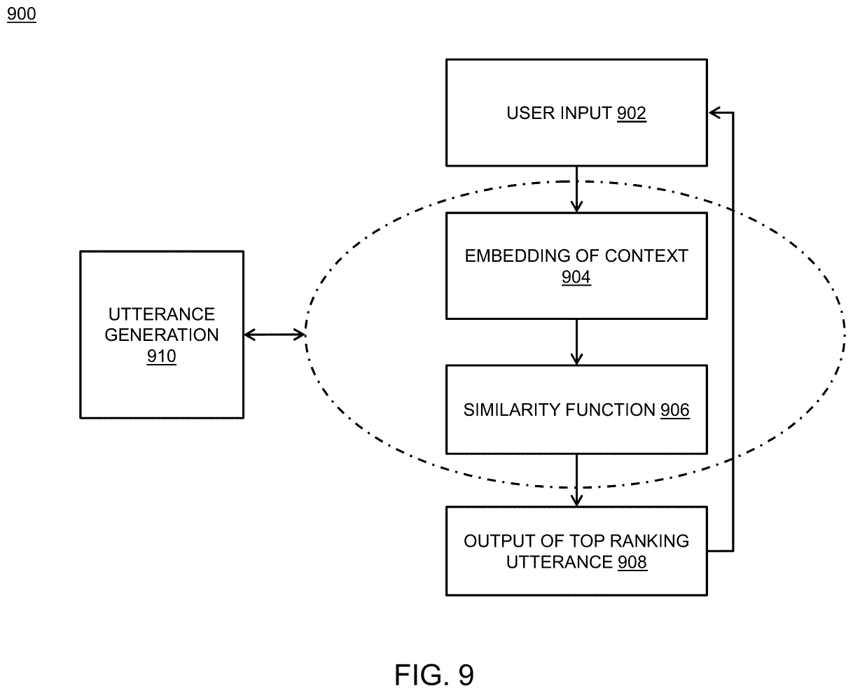

FIG. 9 illustrates a flow chart 900 for depicting a method to provide medical advice to the user in real time based on the medical triage conversation, in accordance with various embodiments of the present disclosure. It may be noted that to explain the flow chart 900, references will be made to the system elements of FIG. 1, and FIG. 2.

FIG. 9 includes a user input 902, an embedding of context 904, a similarity function 906, an output of top ranking utterance 908, and an utterance generation 910. The user input 902 refers to the user input by the user 102. In an example, the user 102 enquires the medical assistant system 206 “What should I eat while suffering from fever?” The medical assistant system 206 generates the embedding of context 904. The medical assistant system 206 performs operations such as cleaning of the user input 902, tokenizing of the user input 902 and the like. Further, the medical assistant system 206 generates the embedding of context 904. The utterance generation 910 is utilized to generate the embedding of context 904. In an embodiment of the present disclosure, the embedding of context 904 includes word embedding, sentence embedding and the like. In an embodiment of the present disclosure, the embedding of context 904 generates separate embedding of the context and separate embedding of the utterance. Furthermore, the medical assistant system 206 applies the similarity function 906 to every pairing of embedding of the context and embedding of the utterance. The output of top ranking utterance 908 is presented to the user 102. (as mentioned above)

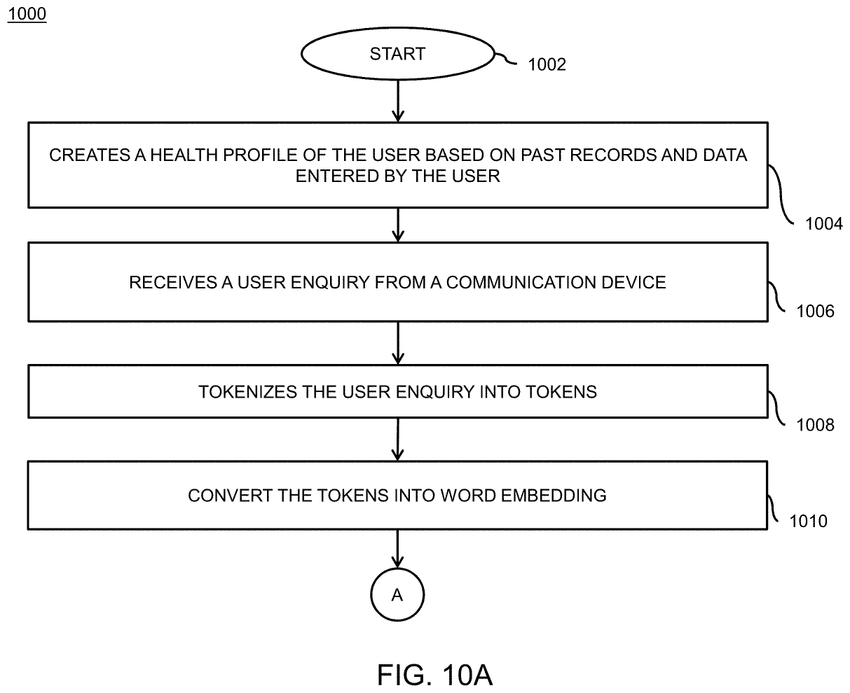

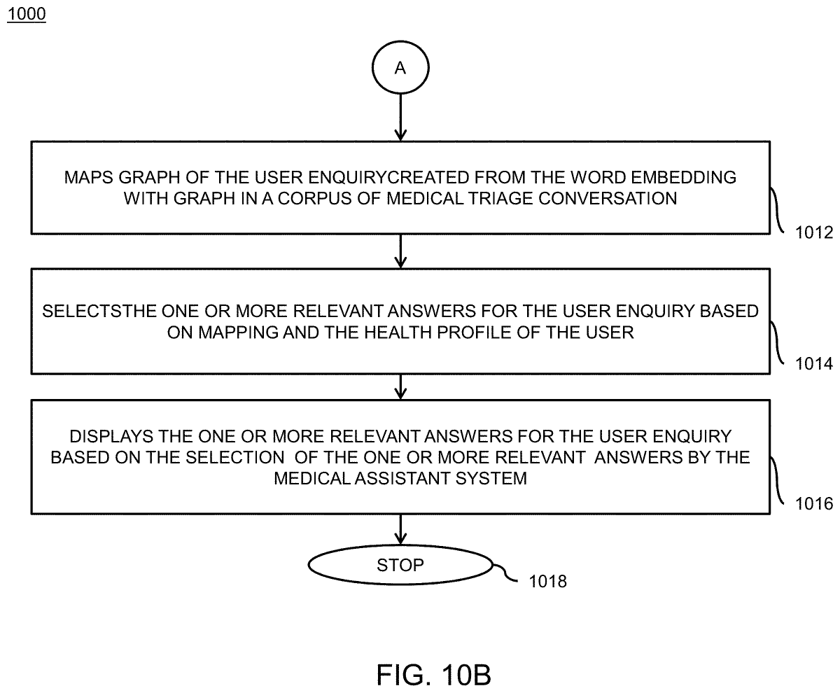

FIGS. 10A and 10B illustrate a flow chart 1000 for answering a user with one or more relevant answers based on the user enquiry, in accordance with various embodiments of the present disclosure. It may be noted that to explain the process steps of flowchart 1000, references will be made to the system elements of FIG. 1 and FIG. 2. It may also be noted that the flowchart 1000 may have lesser or more number of steps.

The flowchart 1000 initiates at step 1002. Following step 1002, at step 1004, creates the health profile of the user 102 based on the past records and data entered by the user 102. At step 1006, the medical assistant system 206 receives the user enquiry from the communication device 104. At step 1008, the medical assistant system 206 tokenizes the user enquiry into the tokens. At step 1010, the medical assistant system 206 converts the tokens into the word embedding. At step 1012, the medical assistant system 206 maps graph of the user enquiry created from the word embedding with graph in the corpus of medical triage conversation. At step 1014, the medical assistant system 206 selects the one or more relevant answers for the user enquiry based on mapping and the health profile of the user 102. At step 1016, the medical assistant system 206 displays the one or more relevant answers for the user enquiry based on the confidence level of the one or more relevant answers selected by the medical assistant system 206. The flow chart 1000 terminates at step 1018.

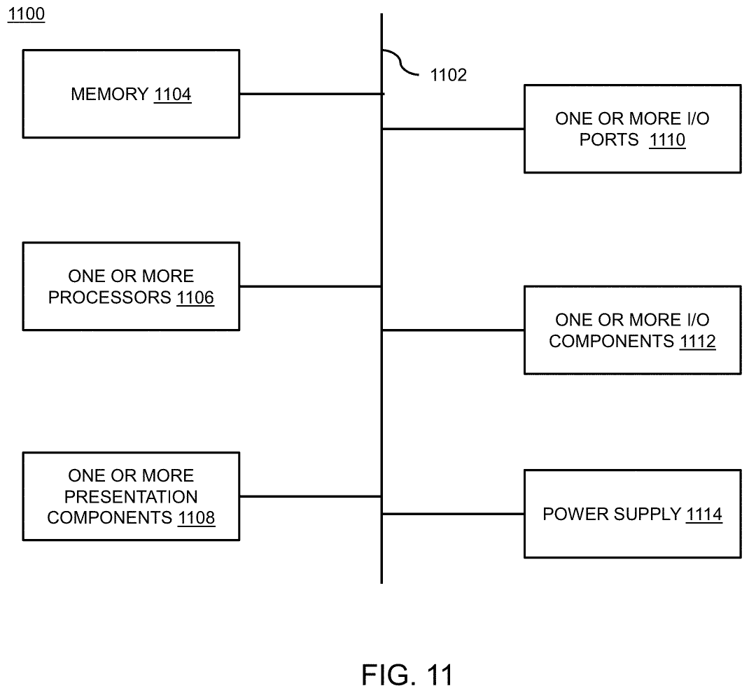

FIG. 11 illustrates a block diagram of a device 1100, in accordance with various embodiments of the present disclosure. In FIG. 11, the device 1100 illustrates internal structural overview of the communication device 104. The device 1100 is a non-transitory computer readable storage medium. The device 1100 includes a bus 1102 that directly or indirectly couples the following devices: memory 1104, one or more processors 1106, one or more presentation components 1108, one or more input/output (I/O) ports 1110, one or more input/output components 1112, and an illustrative power supply 1114. The bus 1102 represents what may be one or more busses (such as an address bus, data bus, or combination thereof). Although the various blocks of FIG. 11 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors recognize that such is the nature of the art, and reiterate that the diagram of FIG. 11 is merely illustrative of an exemplary device 1100 that can be used in connection with one or more embodiments of the present invention. Distinction is not made between such categories as “workstation,” “server,” “laptop,” “hand-held device,” etc., as all are contemplated within the scope of FIG. 11 and reference to “computing device.”

The computing device 1100 typically includes a variety of computer-readable media. The computer-readable media can be any available media that can be accessed by the device 1100 and includes both volatile and nonvolatile media, removable and non-removable media. By way of example, and not limitation, the computer-readable media may comprise computer storage media and communication media. The computer storage media includes volatile and nonvolatile, removable and non-removable media implemented in any method or technology for storage of information such as computer-readable instructions, data structures, program modules or other data. The computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the device 1100. The communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 1104 includes computer-storage media in the form of volatile and/or nonvolatile memory. The memory 1104 may be removable, non-removable, or a combination thereof. Exemplary hardware devices include solid-state memory, hard drives, optical-disc drives, etc. The device 1100 includes the one or more processors 1106 that read data from various entities such as memory 1104 or I/O components 1112. The one or more presentation components 1108 present data indications to a user or other device. Exemplary presentation components include a display device, speaker, printing component, vibrating component, etc. The one or more I/O ports 1110 allow the device 1100 to be logically coupled to other devices including the one or more I/O components 1112, some of which may be built in. Illustrative components include a microphone, joystick, game pad, satellite dish, scanner, printer, wireless device, etc.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and sub-combinations of the various elements, features, functions, and/or properties disclosed herein. The following claims particularly point out certain combinations and sub-combinations regarded as novel and nonobvious. Inventions embodied in other combinations and sub-combinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

What is claimed:

1. A computer-implemented method for providing medical advice to a user in real time based on medical triage conversation, the computer-implemented method comprising:
creating, at a medical assistant system with a processor, a health profile of the user based on past records and data entered by the user;
receiving, at the medical assistant system with a processor, a user enquiry from a communication device;
tokenizing, at the medical assistant system with the processor, the user enquiry into tokens, wherein the tokenization is done to convert text string of the user enquiry into the tokens, wherein the tokenization is done in real time;
converting, at the medical assistant system with the processor, the tokens into word embedding, wherein the conversion is done by converting the tokens into one-hot vector representation which is than fed to recurrent neural network and tanh is applied in real time, wherein the conversion is done to achieve the user embedding of the user enquiry;
mapping, at the medical assistant system with the processor, graph of the user enquiry created from the word embedding with graph in a corpus of medical triage conversation, wherein the mapping is done to identify similar word embedding of the user enquiry in the corpus of medical training dataset related to the user embedding;
selecting, at the medical assistant system with the processor, the one or more relevant answers for the user enquiry based on mapping and the health profile of the user, wherein the one or more relevant answers are correct answer for the user enquiry being selected from the corpus of medical triage conversation, wherein the one or more relevant answers selected comply with one or more protocols for the user enquiry; and
displaying, at the medical assistant system with the processor, the one or more relevant answers for the user enquiry based on the selection of the one or more relevant answers, wherein the one or more relevant answers are displayed in real time on the communication device.

2. The computer-implemented method as recited in claim 1, wherein the corpus of the medical triage conversation comprises a plurality of question-answer pairs, a plurality of medical questions, a plurality of medical articles and a plurality of medical conversations, wherein the corpus of the medical triage conversation is created from one or more sources, wherein the one or more sources comprises medical literature, textbooks, online databases, journal articles, graphics, podcasts, videos, animations and medical data warehouses.

3. The computer-implemented method as recited in claim 1, wherein the past records comprises medical record, prescription, medical history, medical policy detail, hereditary disease, user allergies and user infections, wherein the past records is collected from one or more third party databases.

4. The computer-implemented method as recited in claim 1, wherein the one or more medical protocols comprises rules, regulations and guidelines to provide medical guidance to the user.

5. The computer-implemented method as recited in claim 1, wherein the selection of the one or more relevant answers for the user enquiry is done based on confidence level of each of the one or more relevant answers.

6. The computer-implemented method as recited in claim 1, wherein the health profile of the user comprises name, age, demographic information, medical record, prescription, hereditary disease, allergies, infections, blood group, hemoglobin level, number of platelets and common symptoms.

7. The computer-implemented method as recited in claim 1, wherein the tokenization insert delineation tokens into context and utterances present in the user enquiry, wherein the insertion of the delineation tokens is done to distinguish the one or more relevant answers and the user enquiry.

8. The computer-implemented method as recited in claim 1, further comprising, creating, at the medical assistant system with the processor, the graph of the user enquiry based on the conversion of the user enquiry into the word embedding, wherein the graph is created in real time using artificial intelligence algorithm.

9. The computer-implemented method as recited in claim 1, further comprising, updating, at the medical assistant system with the processor, the user enquiry after receiving the updated user enquiry from the user based on selection from the one or more relevant answers displayed to the user, wherein the updating is done in real time.

10. A computer system comprising:
one or more processors; and
a memory coupled to the one or more processors, the memory for storing instructions which, when executed by the one or more processors, cause the one or more processors to perform a method for providing medical advice to a user in real time based on medical triage conversation, the method comprising:
creating, at a medical assistant system, a health profile of the user based on past records and data entered by the user;
receiving, at the medical assistant system, a user enquiry from a communication device;
tokenizing, at the medical assistant system, the user enquiry into tokens, wherein the tokenization is done to convert text string of the user enquiry into the tokens, wherein the tokenization is done in real time;
converting, at the medical assistant system, the tokens into word embedding, wherein the conversion is done by converting the tokens into one-hot vector representation which is than fed to recurrent neural network and tanh is applied in real time, wherein the conversion is done to achieve the user embedding of the user enquiry;
mapping, at the medical assistant system, graph of the user enquiry created from the word embedding with graph in a corpus of medical triage conversation, wherein the comparison is done to identify similar word embedding of the user enquiry in the corpus of medical training dataset related to the user embedding;
selecting, at the medical assistant system, the one or more relevant answers for the user enquiry based on mapping and the health profile of the user, wherein the one or more relevant answers are correct answer for the user enquiry being selected from the corpus of medical triage conversation, wherein the one or more relevant answers selected comply with one or more medical protocols for the user enquiry; and
displaying, at the medical assistant system, the one or more relevant answers for the user enquiry based on the selection of the one or more relevant answers, wherein the one or more relevant answers are displayed in real time on the communication device.

11. The computer system as recited in claim 11, wherein the corpus of the medical triage conversation comprises a plurality of question-answer pairs, a plurality of medical questions, a plurality of medical articles and a plurality of medical conversations, wherein the corpus of the medical triage conversation is created from one or more sources, wherein the one or more sources comprises medical literature, textbooks, online databases, journal articles, graphics, podcasts, videos, animations and medical data warehouses.

12. The computer system as recited in claim 11, wherein the past records comprises medical record, prescription, medical history, medical policy detail, hereditary disease, user allergies and user infections, wherein the past records is collected from one or more third party databases.

13. The computer system as recited in claim 11, wherein the one or more medical protocols comprises rules, regulations and guidelines to provide medical guidance to the user.

14. The computer system as recited in claim 11, wherein the selection of the one or more relevant answers for the user enquiry is done based on confidence level of each of the one or more relevant answers.

15. The computer system as recited in claim 11, wherein the health profile of the user comprises name, age, demographic information, medical record, prescription, hereditary disease, allergies, infections, blood group, hemoglobin level, number of platelets and common symptoms.

16. The computer system as recited in claim 11, wherein the tokenization insert delineation tokens into context and utterances present in the user enquiry, wherein insertion of the delineation tokens is done to distinguish the one or more relevant answers and the user enquiry.

17. The computer system as recited in claim 11, further comprising, creating, at the medical assistant system, the graph of the user enquiry based on the conversion of the user enquiry into the word embedding, wherein the graph is created in real time using artificial intelligence algorithm.

18. The computer system as recited in claim 11, further comprising, updating, at the medical assistant system, the user enquiry after receiving the updated user enquiry from the user based on selection from the one or more relevant answers displayed to the user, wherein the updating is done in real time.

19. A non-transitory computer-readable storage medium encoding computer executable instructions that, when executed by at least one processor, performs a method for providing medical advice to a user in real time based on medical triage conversation, the method comprising:
creating, at a computing device, a health profile of the user based on past records and data entered by the user;
receiving, at the computing device, the user enquiry from a communication device;
tokenizing, at the computing device, the user enquiry into tokens, wherein the tokenization is done to convert text string of the user enquiry into the tokens, wherein the tokenization is done in real time;
converting, at the computing device, the tokens into word embedding, wherein the conversion is done by converting the tokens into one-hot vector representation which is than fed to recurrent neural network and tanh is applied in real time, wherein the conversion is done to achieve the user embedding of the user enquiry;
mapping, at the computing device, graph of the user enquiry created from the word embedding with graph in a corpus of medical triage conversation, wherein the comparison is done to identify similar word embedding of the user enquiry in the corpus of medical training dataset related to the user embedding;
selecting, at the computing device, the one or more relevant answers for the user enquiry based on mapping and the health profile of the user, wherein the one or more relevant answers are correct answer for the user enquiry being selected from the corpus of medical triage conversation, wherein the one or more relevant answers selected comply with one or more medical protocols for the user enquiry; and
displaying, at the computing device, the one or more relevant answers for the user enquiry based on the selection of the one or more relevant answers, wherein the one or more relevant answers are displayed in real time on the communication device.
